(19) United States
(12) Patent Application Publication
(10) Pub. No.: US 2012/0128667 A1
(43) Pub. Date: May 24, 2012

(54) PENTAMIDINE COMBINATIONS FOR TREATING CANCER

(75) Inventors: Terry Chow, Anjou (CA); Chiaoli Yeh, Anjou (CA); David Griller, Ottawa (CA)

(73) Assignee: ONCOZYME PHARMA INC., Montreal (CA)

(21) Appl. No.: 13/318,197

(22) PCT Filed: May 3, 2010
(86) PCT No.: PCT/IB2010/001012

§ 371 Date: Feb. 10, 2012

Related U.S. Application Data
(60) Provisional application No. 61/174,802, filed on May 1, 2009.
(60) Provisional application No. 61/296,590, filed on Jan. 20, 2010.

(51) Int. Cl. A61K 31/155; A61K 31/7068; A61K 31/4545; A61P 35/04; A61K 31/505; A61K 39/395; A61P 35/00; A61P 35/02; A61K 31/282; A61K 31/519

(52) U.S. Cl. 424/133.1; 514/641; 514/492; 514/49; 514/283; 514/248; 514/274; 424/142.1

(57) ABSTRACT

The present invention relates to the treatment of cancer, e.g., ovarian cancer, breast cancer, pancreatic cancer or colon cancer, with pentamidine and (a) oxaliplatin, (b) gemcitabine, (c) taxol, (d) 5-fluorouracil or (e) CPT 11.

The present invention is related to U.S. Pat. No. 7,115,665, which discloses use of pentamidine to treat cancer. It is incorporated herein in its entirety.

The present invention relates to synergistic combinations of chemotherapeutic agents for treating cancer.

There is a need for agents and combinations thereof that inhibit the proliferation of cancer cells that are less toxic and/or more active than conventional chemotherapeutics, e.g., especially where an agent or combination of agents permits the use of lower dosages of chemotherapeutics administered to cancer patients without loss of therapeutic efficacy.

### SUMMARY OF THE INVENTION

One aspect of the present invention is a method of inhibiting the proliferation of cancer cells comprising administering to a patient in need thereof (1) pentamidine and (2) (a) oxaliplatin, (b) gemcitabine, (c) taxol, (d) 5-fluorouracil or (e) CPT 11 (camptothecin-11, also known as Irinotecan). The agents can be given either separately, for example on consecutive days, or together.

According to another aspect of the present invention, the method inhibits the proliferation of cancer cells and tumour growth.

According to another aspect of the present invention, there is provided a pharmaceutical composition for inhibiting the proliferation of cancer cells and/or tumour growth that comprises a combination of the compounds above. The invention relates to the surprising discovery that the combinations are synergistic.

In a preferred aspect, the cancer cells are squamous cell carcinoma cells, larger cell carcinoma of the lymph node cells, breast cancer cells, colon cancer cells, lung carcinoma cells, melanoma cells, pancreatic cancer cells, leukemia cells, non-small cell lung cancer cells, colon cancer cells, central nervous system (CNS) cancer cells, ovarian cancer cells, renal cancer cells or prostate cancer cells.

In a preferred aspect, the cancer cells are pancreatic cancer cells, colon cancer cells, breast cancer cells or ovarian cancer cells.

In another preferred aspect, pentamidine is combined with gemcitabine, for instance, for treating pancreatic cancer, or is used alone for such purpose; or pentamidine is combined with oxaliplatin, for instance, for treating colon cancer. For the treatment of (advanced or metastatic) breast or ovarian cancer, doxorubicin, 5-fluorouracil, carboplatin, and paclitaxel are examples of components of standard chemotherapy regimens. Capecitabine (Xeloda®), an orally administered systemic pro-drug of 5′-deoxy-5-fluorouridine (5′DFUR) which is converted to 5-fluorouracil, is also used. While these treatments have extended survival, patients eventually experience disease progression. The incorporation of pentamidine in combination with standard chemotherapy, for example, doxorubincin or 5-fluorouracil or carboplain or paclitaxel, comprises another aspect of this invention.

Pentamidine refers to the free compound or to the compound in salt form, e.g., as the commercially available pentamidine isethionate, or any other pharmaceutically acceptable salt.

The present invention also relates to the further combination of the above agent combinations with additional agents that cause DNA breaks. Including these types of agents provides a valuable tool for cancer therapy. Agents that induce DNA breaks that are within the scope of the present invention include but are not limited to cisplatin, mitomycin C, melphalan, carmustine, adriamycin, taxol, 5-fluorouracil, bevacizumab, capecitabine, folinic acid (also known as leucovorin), ionizing irradiation and bleomycin or with any agent 2(a), 2(b) or 2(c) not in the above combination. Without wishing to be bound by theory, such combinations are believed to operate in view of the inhibition of endo-exonuclease activity by pentamidine. (Other endo-exonuclease activity inhibitors can also be used together with or in place of pentamidine, such as distamycin A and berenil). Such inhibition prevents repair of double-breaks induced directly or indirectly by the mentioned DNA break-inducing agents. The mentioned DNA break-inducing agents can cause double strand breaks directly or can cause single strand breaks that progress to double strand breaks. This is a common occurrence in biological systems. The endo-exonuclease inhibitors such as pentamidine prevent double break repair and thus enhance anticancer effects.

Compositions or mixtures of the disclosed compound combinations may be administered to patients, which include humans and animals. Such compositions or formulations are conventionally prepared. Compositions include all pharmaceutical formulations of a compound and a compound in its pure state. Combinations can include two or more compositions of the individual agents. These include two or more different formulations of a compound such as a tablet formulation for one agent and a liquid formulation for another. Mixtures of two or more compounds in the same formulation are also within the scope of the invention. Compositions also include the usual conventional adjuvants/excipients well known in the pharmaceutical field.

Pharmaceutical formulations can thus be adapted for administration via any desired suitable method, preferably by fully conventional methods, for example by oral (including buccal or sublingual), rectal, nasal, topical (including buccal, sublingual or transdermal), vaginal or parenteral (including subcutaneous, intramuscular, intravenous or intradermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for example, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, calcium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinylpyrrolidone, a dissolution retardant, such as, for example, paraffin, an absorption accelerator, such as, for example, a quaternary salt, and/or an absorbent, such as, for example, bentonite, kaolin or dicalcium phosphate. The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The compounds according to the invention can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compound. Syrups can be prepared by dissolving the compound in an aqueous solution with a suitable flavour, while elixirs are prepared using a non-toxic alcoholic vehicle. Suspensions can be formulated by dispersion of the compound in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in the freeze-dried (lyophilised) state, so that only the addition of the sterile carrier liquid, for example water for injection purposes, immediately before use is necessary.

The individual agents that comprise the combinations can be administered to the patient at the same time or at different times depending upon their bioavailability and toxicity. Their packaging into kits for administration to the patient also forms part of this invention. The agents can be formulated in a single pharmaceutical composition or can be separately formulated.

Pharmaceutical compositions of the above combinations are used to treat patients having cancer. Vehicles for delivering the compounds of the present invention to target tissues throughout the human body include saline and D5W (5% dextrose and water). Excipients used for the preparation of oral dosage forms of the compounds of the present invention include additives such as a buffer, solubilizer, suspending agent, emulsifying agent, viscosity controlling agent, flavor, lactose filler, antioxidant, preservative or dye. There are conventionally preferred excipients for parenteral and other administration. These excipients include serum albumin, glutamic or aspartic acid, phospholipids and fatty acids.

Formulations can be in liquid form stored in a vial or an intravenous bag. The compounds of the present invention may also be formulated in solid or semisolid form, for example pills, tablets, creams, ointments, powders, emulsions, gelatin capsules, capsules, suppositories, gels or membranes.

The preferred route of administration is intravenous. Other acceptable routes of administration include oral, topical, rectal, parenteral (injectable), local, inhalant and epidural administration. The compositions of the invention may also be conjugated to transport molecules or included in transport modalities such as vesicles, micelles, and polymers to facilitate transport of the molecules. Methods for the preparation of pharmaceutically acceptable compositions that can be administered to patients are known in the art.

The compositions of the invention may also be conjugated to transport molecules, monoclonal antibodies or transport modalities such as vesicles and micelles that preferentially target cancer cells or that potentiate cancer cells to receive drugs.

Pharmaceutical compositions including the compounds of the present invention can be administered to humans or animals. Dosages to be administered also conventionally depend on individual patient condition, indication of the drug, physical and chemical stability of the drug, toxicity, the desired effect and on the chosen route of administration (Robert Rakel, ed., Conn's Current Therapy (1995, W.B. Saunders Company, USA)).

Excipients can also include components such as micelles, vesicles and liposomes that enhance the therapeutic performance of the compound and other agents. The action of vesicles, micelles and liposomes includes improving the solubilization of the compounds and agents, improving their delivery to tumour cells, and interacting with tumour cells to make these cells more permeable to compounds and agents. Improving efficiency could improve treatment or allow equivalent results with reduced dosing and side-effects.

Typical doses for each of the agents for use in this invention are in the normal ranges conventionally known for each known agent used individually to treat cancer. For pentamidine, typical doses are 2-8 mg/kg body weight in humans. These amounts can be lowered per this invention due to synergistic effects in the combinations. Typical dose ranges for each agent in the combinations are: pentamidine 2-8 mg/kg body weight in humans; gemcitabine 800-1250 m g/m² of surface area in humans; CPT 11 75-350 mg/m² of surface area in humans; and oxaliplatin 85-130 mg/m² of surface area in humans. Doses can be lowered from the amounts in these ranges typically by 10 to 50% due to synergism.

Regimens (e.g. timing of doses, durations, etc.) are conventionally determinable with the guidance of conventional usage of these agents individually.

In the case of pentamidine, for example, guidance may be obtained from a study of patients given 180 to 200 mg of pentamidine in a 2-hour infusion. It showed that levels in the bloodstream go down rapidly over a few hours; and that the kidneys excrete only 7 mg of pentamidine into the urine in the first 24 hours (Conte, J. E., Jr.: J. Infect. Diseases (1991), 163, 169). Since pentamidine is not readily metabolized in the liver, almost all of the material is distributed from the blood stream to body tissue where it stays. In addition, the amount found in the urine does not increase significantly with repeated dosing. This means that when pentamidine is given repeatedly, it accumulates in body tissues. Pentamidine was detected in tissue 25 days after final dose. Hence, pentamidine is only slowly released from tissue. It is also widely distributed in tissue (Goa, K. L., Campoli-Richards, D. M.; Drugs (1987), 33, 242). Thus, pentamidine can be administered to the patient before, after, or concurrently with other chemotherapy since its effectiveness depends on its distribution to and persistence in body tissues over long periods.

The way in which other chemotherapy agents are used in conjunction with pentamidine depends on their pharmacological characteristics. Thus, a convenient mode of dosing is to take the normal cycle of administration of a chemotherapy drug and to precede it with administration of pentamidine. This may be illustrated in conjunction with the combination of, for example, cis-platinum used effectively in combination with pentamidine to control cancer growth. Cis-platinum reacts slowly with water in the body to give an active form that binds to tissue. If it is injected slowly into patients urinary excretion can be as high as 75%. Therefore, rapid dosing is often used to ensure that the kidneys cannot excrete the drug before it is distributed to body tissue (Belt, R. J., Himmelstein, K. J., Patton, T. F., Bannister, S. J., Sternson, L. A., Repta, A. J., Cancer Treatment Rep. (1979), 63, 1515). Thus, when pentamidine is used in conjunction with cis-platinum, a prudent approach is to give pentamidine to the patient a day before cis-platinum so that the kidneys are not over burdened by the administration of the two drugs. If two doses of pentamidine are needed, the first can be given two days before cis-platinum (day −2) and the second can be given one day before cisplatinum (day −1).

Often in oncology combinations of drugs are used. In colon cancer, for instance, examples include the administration of oxaliplatin, 5-fluorourocil, and leucovrin “FOLFOX” or irinotican, 5-fluorouracil and leucovorin “FOLFIRI”. These combinations are typically administered to the patient every two weeks. Thus, when pentamidine is added to therapy, it can conveniently be given one or two days before standard chemotherapy. However, since pentamidine persists in body tissue, it can be as effective if given several days before standard chemotherapy.

A further example relates to human pancreatic cancer. Here, a typical treatment cycle involves administration of gemcitabine 800-1250 mg/m² of surface area once a week for three weeks followed by a week of rest. When pentamidine is used together with gemcitabine it can conveniently be administered during the first week of the cycle on day −2 and day −1 prior to the administration of gemcitabine.

As a further example of suitable pentamidine dosing for use in combination with other cancer regimens, pentamidine can be given to patients intravenously in the following doses prior to such chemotherapy:

| | Day −2 | Day −1 |
|---|---|---|
| | Dose (mg/kg) | Dose (mg/kg) |
| Option 1 | | 4 |
| Option 2 | 4 | 4 |
| Option 3 | | 5 |
| Option 4 | 5 | 5 |
| Option 5 | | 6 |
| Option 6 | 6 | 6 |

Moreover, dosing for a patient can be either escalated from lower to higher options or reduced in, respectively, the absence or presence of side-effects and, as is conventional, following the advice of the treating physician. Because pentamidine accumulates in body tissue, as discussed, it can be administered at any time in the cycle of normal chemotherapy, i.e., dosing is not limited to day-1 and day-2. Optimal dosing can be routinely determined.

Since pentamidine has a side-effect profile and mechanism of action that is quite different to those of standard anticancer agents, it can be used in combination with them without inducing adverse drug reactions that are substantially worse than those induced by the drugs when used alone. Given the life-threatening nature of many cancers, patients are treated aggressively with chemotherapy. Treatment in conjunction with pentamidine can be given until side-effects of the standard chemotherapy agent become evident. At this point, administration of the standard chemotherapy agent can be halted and therapy with pentamidine alone can be continued. The sustained use of pentamidine can be of benefit to patients since pentamidine is an effective anticancer agent in its own right. Reasonable doses of pentamidine to be used either in combination therapies or in mono-therapy are 6 mg/kg of body weight or 4 mg/kg of body weight.

Pentamidine has side-effects of its own, the most significant of which in this context is the possibility that patients might suffer pancreatitis. This side-effect can be pronounced if pentamidine is administered for many consecutive days, e.g., 10 to 15 at doses of 4-6 mg/kg/day as is the case when it is used to treat parasitic diseases. However, in the dosing schedules described herein where one or two doses may be given every two weeks, the risks of pancreatitis are greatly reduced. If pancreatitis occurs, pentamidine administration can be stopped until the patient recovers but standard chemotherapy may be continued in the interim. Sustained use over many days with lower doses of pentamidine, e.g., 1-4 mg/kg per day, affords another means of reducing toxicity while maintaining efficacy.

As in all therapies, treating physicians have to consider the characteristics and use of drugs in light of the patients' physical condition and symptoms and administration has to be routinely modulated accordingly.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

The entire disclosure[s] of all applications, patents and publications, cited herein are incorporated by reference herein.

The preceding examples can be repeated with similar success by substituting the generically or specifically described reactants and/or operating conditions of this invention for those used in the preceding examples.

### EXAMPLES

### Example 1

### Purpose:

Synergistic effect in anticancer therapy was generated by using pentamidine in combinations with each of the following: taxol, oxaliplatin, gemcitabine, or CPT 11.

### Method:

Cell Survival-MTT assay: The MTT (3-[4,5-Dimethylthiazol-2-yl]-2,5 diphenyl tertrazolim bromide) method of determining cell growth/cytotoxicity was used to determine cell survival. MTT is a tetrazolium salt that binds to b mitochondrial dehydrogenases of living cells. Binding converts yellow, water soluble MTT to an insoluble, purple formazan crystal. The crystals are solubilized with a 50% N,N-dimethylformamide (vol/vol), 20% SDS (wt/vol) solution (pH 4.7), and absorbance is determined at a wavelength of 570 nm. Unbound MTT is not detectable at this wavelength. The amount of bound MTT measured in the assay is proportional to the number of live cells present. (Niks and Otto 1990: “Towards an optimized MTT assay,” J. Immunol. Methods. 130, 149-151, Hussain et al. 1993; “A new approach for measurement of cytotoxicity using colorimetric assay,” J. Immunol. Methods. 160, 89-96).

Cells were harvested from cell cultures using the standard protocol (Trypsin/EDTA). The cells (1000 to 5000 cells in 50 μl of solution depending on cell type used) were then plated and incubated overnight at 37° C. before the addition of the agent or combination of agents.) After 2 days of incubation at 37° C., 10 μl of a 5 mg/ml solution of MTT was then added to all the wells and to a media control well. The plates were further incubated for 4 hours. A 100 μl of MTT solubilization buffer was added and the plates were incubated overnight at 37° C. The plates were then read on the ELISA plate reader with absorbance at 570 nm and a reference at 630 nm.

The effects of combinations were tested on three representative cancer cell lines: H661 (lung cancer (carcinoma)), MCF-7 (breast cancer (adenocarcinoma, pleural effusion)), and HT29 (colon cancer (adenocarcinoma, primary tumor)). Initial assays were carried out to determine the concentrations at which taxol, oxaliplatin, gemcitabine, or CPT 11 (also known as irinotecan) killed approximately 10% of the cells under investigation. In a second series of assays, pentamidine was added to the cell cultures. Several concentrations of pentamidine were tested in combination with each of taxol, oxaliplatin, gemcitabine, or CPT 11 and the LC₅₀ was determined i.e. the concentration of pentamidine that killed 50% of the remaining cells.

The addition of pentamidine to a sub-lethal dose of cytotoxic chemotherapeutic agents greatly increased the anticancer effect (from 2-fold to 50-fold) for breast cancer (MCF-7), lung cancer (H661) and colon cancer (H T29) cells, as shown in Table 1.

TABLE 1
LC₅₀ of Pentamidine On Cancer Cells When Used Alone Or In Combination
With Taxol, Oxaliplatin, Gemcitabine, or CPT 11

| | | | Pentamidine | | Pentamidine |
|---|---|---|---|---|---|
| | | Pentamidine | (mM) With | Pentamidine | (mM) With |
| Cancer | Pentamidine | (mM) with | Oxaliplatin | (mM) With | Gemcitabine |
| cell type | (mM) 2 days¹ | Taxol (2 μM) | (0.25 μM) | CPT11 (0.25 μM) | (0.26 μM) |
| H661 | 0.15 | 0.007 | 0.003 | 0.003 | 0.027 |
| MCF-7 | 0.15 | 0.06 | 0.009 | 0.026 | 0.066 |
| HT29 | 0.27 | 0.13 | 0.009 | 0.012 | 0.030 |

¹Length of exposure to mixture.

Since the concentration of the cytotoxic agents killed 10% of the cells when used alone, an additive effect would simply have manifested itself as a small improvement in the performance of pentamidine, roughly corresponding to an improvement of about 10% versus that of pentamidine alone. The data show that the combinations allowed the concentration of pentamidine to be reduced by 100% (HT29 with taxol—worst case examined) and by 5000% (oxaliplatin or gemcitabine with H661—best cases examined) while maintaining the same cell killing efficiency. All the cytotoxic agents displayed a strong synergistic effect when used in combination with pentamidine.

This effect is also demonstrated by the data in Tables 2A-C, where pentamidine and various cytotoxic agents are used at higher concentrations than those described above. The extent to which each kill cells when used alone is reported in the tables. The data are followed by the extent of cell death when the compounds are used in combination. Again the combinations show synergy and not simple additivity.

TABLE 2A
Percentage of cells killed at various drug concentrations

| | Pentamidine | CPT-11 | Pentamidine (0.15 mM) + |
|---|---|---|---|
| Cell line | (0.15 mM) | (3.2 μM) | CPT-11 (3.2 μM) |
| H661 | 50% | 14% | 93% |
| MCF7 | 50% | 30% | 100% |
| HT29 | 13% | 6% | 90% |

TABLE 2B

| | Pentamidine | Oxaliplatin | Pentamidine (0.15 mM) + |
|---|---|---|---|
| Cell line | (0.15 mM) | (0.25 μM) | Oxaliplatin (0.25 μM) |
| H661 | 50% | 14% | 95% |
| MCF7 | 50% | 8% | 71% |
| HT29 | 13% | 10% | 93% |

TABLE 2C

| | Pentamidine | Gemcitabine | Pentamidine (0.15 mM) + |
|---|---|---|---|
| Cell line | (0.15 mM) | (0.26 μM) | Gemcitabine (0.26 μM) |
| H661 | 50% | 25% | 100% |
| MCF7 | 50% | 23% | 100% |
| HT29 | 13% | 23% | 100% |

### Example 2

### Clinical Trial Pancreatic Cancer

A non-randomized, open label, Phase I/IIa clinical trial is designed to assess the effect of intravenous (I.V.) pentamidine for subjects with advanced or metastatic pancreatic cancer undergoing standard chemotherapy (gemcitabine regimen).

A total of 15-20 subjects with pancreatic cancers are being enrolled over a period of 12 months. Pentamidine is being administered I.V. over a period of 1-2 hours, in a continuous regimen, with a starting dose of 6 mg/kg of body weight of pentamidine isethionate. Pentamidine is being administered two days prior (Day −2) to the start of a 21-28 day standard chemotherapy cycle for pancreatic cancer. A further dose is being given on Day −1.

All subjects are being given a standard of care chemotherapy regimen. Subjects continue treatment as long as they receive clinical benefit, or until objective disease progression is documented, or until they withdraw from the trial for other reasons.

### Example 3

### Clinical Trial Colon Cancer

A non-randomized, open label, Phase I/IIa clinical trial is designed to assess the effect of I.V. pentamidine for subjects with metastatic colon cancer undergoing second-line chemotherapy (modified FOLFOX-6 (mFOLFOX6), or Capecitabine and Oxaliplatin, or FOLFIRI or IROX, or Capecitabine and Irinotecan containing regimens) treatment and/or chemotherapy as per physician choice for third line and above treatment regimen. (FOLFOX regimens contain oxaliplatin, FOLFIRI regimens contain CPT 11 also known as Irinotecan; and IROX regimens contain Irinotecan and Oxaliplatin). Patients may also receive bevacizumab (Avastin) as part of chemotherapy or cetuximab (Erbitux) or panitumumab (Vectibix). Twenty-two patients are enrolled to date.

Pentamidine is being administered two days prior (Day −2) to the start of a 14 day standard chemotherapy cycle for metastatic colon cancer. A further dose is being given on Day −1. Pentamidine is being administered I.V. over a period of 1-2 hours, in a continuous regimen, with a starting dose of 4 mg/kg of body weight of pentamidine isethionate.

The study design allows for dose escalation to 6 mg/kg of pentamidine and for continuing patients on pentamidine alone when side effects from the other anticancer agents become pronounced. Both dose escalation and treatment with pentamidine alone are at the discretion of the treating physician.

The following combinations with pentamidine are tested in patients: FOLFOX (fluorouracil, folinic acid and oxaliplatin) or modified versions thereof with or without bevacizumab, FOLFIRI (fluorouracil, folinic acid and irinotecan) or modified version thereof with or without bevacizumab, CPT-11 with or without bevacizumab, CPT-11 with or without oxaliplatin, and capecitabine. Almost all patients will have previously failed on their current treatment or a combination thereof.

Interim results demonstrate that pentamidine significantly enhances overall survival when compared with best current therapy.

### Example 4

### Clinical Trial Breast and Ovarian Cancer

A non-randomized, open label, Phase I/IIa clinical trial is designed to assess the effect of I.V. pentamidine for subjects with breast and/or ovarian tumors and/or metastases derived from breast and/or ovarian tumours. Patients are receiving pentamidine beginning with two doses of pentamidine isethionate (6 mg/kg) prior to each cycle of standard chemotherapy.

Pentamidine is being administered two days prior (Day −2) to the start of a standard chemotherapy cycle for breast and/or ovarian cancer. A further dose is being given on Day −1. Pentamidine is being administered I.V. over a period of 1-2 hours, in a continuous regimen.

For the treatment of localized or metastatic breast or ovarian cancer, doxorubicin, 5-fluorouracil, carboplatin, and paclitaxel are examples of components of standard chemotherapy regimens. Capecitabine (Xeloda®), an orally administered systemic prodrug of 5′-deoxy-5-fluorouridine (5′DFUR) which is converted to 5-fluorouracil, is also used. The incorporation of pentamidine in combination with standard chemotherapy, for example, doxorubincin or 5-fluorouracil or carboplain or paclitaxel, comprises another aspect of this invention.

The study design allows for continuing patients on pentamidine alone when side effects from the other anticancer agents become pronounced. Dose escalation, reduction, and treatment with pentamidine alone are at the discretion of the treating physician.

### Example 5

### Phase I/II Trial of Pentamidine with Fluorouracil, Oxaliplatin and/or CPT-11 Containing Chemotherapy in Patients with Previously Treated Metastatic Colorectal Cancer (mCRC)

### Introduction

Colorectal cancer is the 2^{nd} leading cause of cancer death in North America
Combination chemotherapy with biologic agents has extended median survival in patients (pts) with mCRC to approximately 24 months
Novel agents are being actively investigated
Endo-exonuclease (EE), a key enzyme in DNA recombination and repair, has been show to be overexpressed in cancer cells^{1,2,3}
Pentamidine inhibits EE and has been shown to have disease stabilizing activity in metastatic cancer^{1,4}
In vitro studies have shown that pentamidine can potentiate the effects of cytotoxic chemotherapy on malignant cells; by impairing their capacity for DNA repair, they are more susceptible to DNA damaging agents^{1,2,3}

### Study Objectives

To evaluate the safety and efficacy of combining pentamidine with fluoropyrimidine, oxaliplatin and/or CPT-11 containing chemotherapy (CTX) in pts with mCRC who have failed prior lines of standard treatment
Primary endpoints: treatment safety and tolerability
Secondary objectives: response rate (RR), progression-free survival (PFS) and overall survival (OS)

### Methods

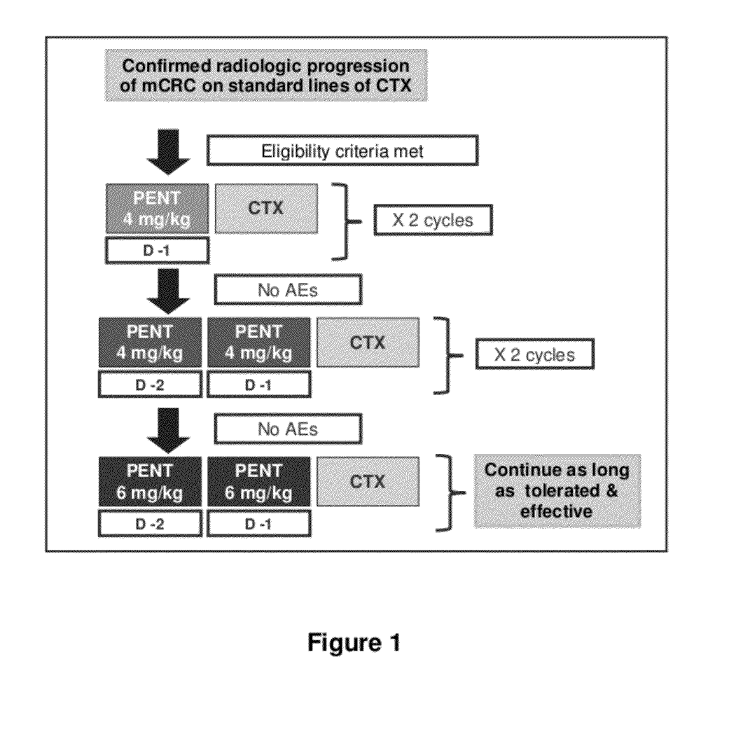

Eligibility criteria: radiologic evidence of progression of mCRC on ≧1 prior lines of standard CTX; ≧18y/o; ECOG 0-2; normal EKG; adequate hematologic, hepatic and renal function; life expectancy>3 months; informed consent
Pentamidine at 4 mg/kg was begun the day before CTX and gradually escalated to a maximum dose of 6 mg/kg for 2 consecutive days before CTX (see FIG. 1)
CDC was chosen by the patient's treating oncologist.
Adverse events (AEs) were graded according to the NCI CTCAEv3 classification system
Dose limiting toxicity (DLT)=any grade 3 or 4 occurring within the first 2 cycles of treatment that can be attributed to pentamidine
Maximum dose of pentamidine chosen for this study was 6 mg/kg for 2 consecutive days before CTX; higher doses were not tested
Screening CT chest/abdomen/pelvis within 28 days of starting study treatment->repeat q3 cycles or as per standard of care
Radiologic response was assessed according to RECIST criteria
An extension phase was opened for pts with no disease progression after 6 cycles of pentamidine

### Results: Clinical Characteristics

Preliminary results on the initial 17 patients enrolled in this ongoing phase I/II trial are presented (Table 3)
Median age of first 17 pts=66 (range 43-82)
Median treatment duration of first 17 pts=15 weeks (range 0.3-58)

TABLE 3
Patient characteristics of initial 17 pts

| Median patient age | 66 years (43-82) | — |
|---|---|---|
| Male:Female ratio | 10:7 | 59%:41% |
| ECOG status: | | |
| 0 | 10 | 59% |
| 1 | 5 | 29% |
| 2 | 2 | 12% |
| Number of previously | | |
| failed CTX lines: | | |
| 1 | 3 | 18% |
| 2 | 7 | 41% |
| 3 or more | 6 | 35% |
| Data pending | 1 | 6% |
| Current CTX (selected | | |
| by treating oncologist) | | |
| Irinotecan-based (with | 8 (2) | 47% (12%) |
| Bevacizumab) | | |
| Oxaliplatin-based (with | 4 (1) | 24% (6%) |
| Bevacizumab) | | |
| Irinotecan and | 4 (1) | 24% (6%) |
| Oxaliplatin-based | | |
| Other | 1 | 6% |
| Initial dose of | | |
| pentamidine prior to | | |
| CTX: | | |
| 1 × 4 mg/kg | 3 | 18% |
| 2 × 4 mg/kg | 12** | 71%** |
| 2 × 6 mg/kg | 2 | 12% |

Note:
A failed line of CTX = disease progression during or within 6 months of CTX end. Failure of CTX due to toxicity was not counted as CTX failure in this study. Numbers may not add up to 100% due to rounding.
*Study and data collection have not closed.
**One patient was mistakenly administered 5 mg/kg instead of 4 mg for the first six weeks (3 cycles) of treatment.

### Results: Adverse Events

13 out of 17 pts were evaluable for preliminary safety and tolerability analysis (Table 4). Data pending for 4 pts
Grade 3/4 AEs attributed to pentamidine were hyperglycaemia (23%) and hyperlipasemia (15%).
   NB. Concomitant drugs (e.g. decadron), CTX preparation in D5W and/or inclusion of pts with type 2 diabetes may confound direct attribution of hyperglycaemia to pentamidine
DLT were anorexia and hyperglycaemia, which each occurred in 8% of pts
Toxicity was consistent with known side effects of pentamidine

TABLE 4
Major Adverse Events (preliminary analysis)*

| Adverse Event | | | Tx | | | | |
|---|---|---|---|---|---|---|---|
| Category and | Patient | AE | Cycle | Pentamidine | Attribution | | |
| Type | ID | Grade | No. | dose | Pentamidine | CTX | Consequence |

CNS:

| | | | | | | | |
|---|---|---|---|---|---|---|---|
| Fainting/syncope | 8 | 3 | 18 | 4 mg/kg × 1 | No | no | Tx delayed |

Constitutional:

| | | | | | | | |
|---|---|---|---|---|---|---|---|
| Fatigue | 5 | 4 | 4 | 4 mg/kg × 2 | No | no | discontinued |
| | | | | | | | study |
| | 7 | 3 | 14 | 4 mg/kg × 1 | possibly | possibly | continued |
| | | | | | | | study |
| General | 5 | 4 | 4 | 4 mg/kg × 2 | No | no | discontinued |
| deterioration | | | | | | | study |

Dermatologic:

| | | | | | | | |
|---|---|---|---|---|---|---|---|
| Hand-foot | 3 | 3 | 2 | 4 mg/kg × 2 | No | definitely | Tx delayed |
| reaction | | | | | | | |

Gastrointestinal:

| | | | | | | | |
|---|---|---|---|---|---|---|---|
| Anorexia | 5 | 3 | 2 | 4 mg/kg × 2 | possibly | possibly | continued |
| | | | | | | | study |
| Diarrhea | 5 | 3 | 3 | 4 mg/kg × 2 | No | definitely | continued |
| | | | | | | | study |
| | 10 | 3 | 5 | 4 mg/kg × 2 | possibly | possibly | Tx delayed |
| | 12 | 3 | 3 | 4 mg/kg × 2 | No | probably | continued |
| | | | | | | | study |

Genitourinary:

| | | | | | | | |
|---|---|---|---|---|---|---|---|
| Urinary tract | 11 | 3 | 2 | 4 mg/kg × 2 | No | no | Tx delayed |
| infection | | | | | | | |

Hematologic:

| | | | | | | | |
|---|---|---|---|---|---|---|---|
| Neutropenia | 1 | 3 | 5 | 4 mg/kg × 1 | possibly | definitely | continued |
| | | | | | | | study |

Metabolic:

| | | | | | | | |
|---|---|---|---|---|---|---|---|
| Hyperglycaemia | 1 | 3 | 1 | 4 mg/kg × 1 | possibly | no | continued |
| | | | | | | | study |
| | 11 | 4 | 8 | 4 mg/kg × 2 | definitely | no | discontinued |
| | | | | | | | study |
| | 12 | 3 | 10 | 4 mg/kg × 2 | definitely | possibly | discontinued |
| | | | | | | | study |
| Elevated Lipase | 7 | 3 | 14 | 4 mg/kg × 1 | posssibly | possibly | Tx delayed |
| | 8 | 3 | 19 | 4 mg/kg × 1 | definitely | no | discontinued |
| | | | | | | | study |

Note:
Data pending on 4 pts.
Grey text = adverse events not attributed to pentamidine.
Tx = therapy.
*Study and data collection have not closed.

### Results: Clinical Outcomes

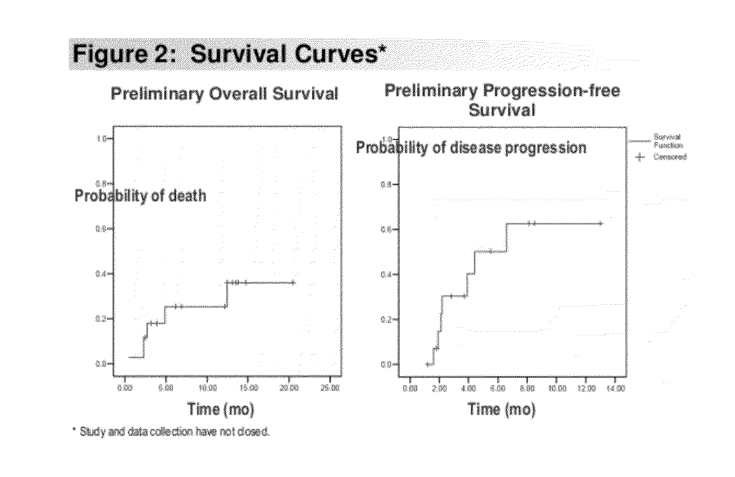

14 out of 17 patients were evaluable for response (Table 5)
35% of pts had SD and 47% had PD at patient exit
Preliminary analysis of the median PFS time=4.4 months (FIG. 2)
Median OS time has not yet been reached
Changes in CEA did not correlate with response (data not shown)

TABLE 5
Preliminary Clinical Outcomes*
Clinical outcomes of initial 17 pts

| Median duration of treatment | 15.1 wk (0.3-58.4) | — |
|---|---|---|
| Best response during treatment*: | | |
| CR | 0 | 0% |
| PR | 1 | 6% |
| SD | 10 | 59% |
| PD | 4 | 24% |
| Unevaluable (Data pending) | 3 (1) | 18% (6%) |
| Response at patient exit from | | |
| trial**: | | |
| CR | 0 | 0% |
| PR | 0 | 0% |
| SD | 6 | 35% |
| PD | 8 | 47% |
| Unevaluable (Data pending) | 3 (1) | 18% (6%) |
| Reasons for patient exiting the | | |
| trial: | | |
| Toxicity attributed to pentamidine | 3 | 18% |
| PD | 6 | 35% |
| Fatigue | 2 | 12% |
| Other (general deterioration, | 3 | 18% |
| surgery, jaundice) | | |
| Data pending | 3 | 18% |

Note:
CR = complete regression,
PR = partial regression,
SD = stable disease,
PD = progressive disease.
Numbers may not add up to 100% due to rounding.
*Study and data collection have not closed.
**Best response indicates best tumour response achieved as determined by CT scan.

### Conclusions

Toxicity associated with the combination of pentamidine and CTX was consistent with that observed in the literature and was manageable
Pentamidine & CTX appears to have disease stabilizing activity in mCRC that has progressed on standard lines of treatment

### References

Chow T Y, Alaoui-Jamali M A, Yeh C et al. The DNA double-stranded break repair protein endo-exonuclease as a therapeutic target for cancer. Mol Cancer Ther 2004; 3(8):911-9.
Sibgat A. Choudhury, and Terry Y-K. Chow, DNA repair protein: The endo-exonuclease as a new front in cancer therapy. Future Oncology 1(2):265-271, 2005.
Choudhury S A, Kauler P, Devic S et al. Silencing of endo-exonuclease expression sensitizes mouse B 16F10 melanoma cells to DNA damaging agents. Invest New Drugs 2007; 25(5):399-410.
von Hoff D, Gorton M, Turner J et al. A phase I study with CRx-026, a novel dual action agent, in patients with advanced solid tumors. J Clin Oncol, 2005 ASCO Annual Meeting Proceedings. Vol 23, No. 16S, Part I of II (June 1 Supplement), 2005: 3073

From the foregoing description, one skilled in the art can easily ascertain the essential characteristics of this invention and, without departing from the spirit and scope thereof, can make various changes and modifications of the invention to adapt it to various usages and conditions.

### Example 6

### Human Xenograph Study

A human xenograph study in a mouse model was conducted to demonstrate the anti tumor activity of Pentamidine administered intraperitoneally twice a week in combination with Gemcitabine administered intraperitoneally twice a week in the BxPC3 human pancreas xeongraft model on CB 17 SCID female mice.

BxPC3 cells were transplanted subcutaneously into the flank of each animal as a suspension of tumor cells (5×10⁶ cells in 0.1 mL in PBS) on Jan. 4, 2010 (day 1). Transplantation was performed under a laminar airflow hood. Four (4) days after BxPC3 cell injections, mice were randomized (day treatment began) into 4 groups of 10 mice each based on tumor size so that the average tumor size in each group was comparable. Five (5) mice were rejected of this study because no tumor grew, tumors were too small, or tumors were too big. Animals were labeled using the “ear punching” method so that for each group, all 10 animals could be distinguished. Each group of 10 mice was housed in 2 separate cages of 5 mice each; animal numbers 1 to 5 were housed in Cage A and animal numbers 6 to 10 were housed in Cage B.

Prior to every dosing injection, each animal was weighed and received their respective formulations. Mice in group 1 were treated intraperitoneally for two consecutive days, stop one day and two other consecutive days for nine weeks (one mouse reached one end points) by direct injection in the abdominal cavity with 0.9% NaCl usp. Mice in group 2 were treated intraperitoneally, bi-weekly (Monday and Thursday) at 45 mg/kg with Pentamidine for nine weeks. Mice in group 3 were treated intraperitoneally, bi-weekly (Tuesday-Friday) at 150 mg/kg with Gemcitabine for eleven weeks. Mice in group 4 were treated first with Pentamidine administered intraperitoneally bi-weekly (Monday and Thursday) at 45 mg/kg and with Gemcitabine administered intraperitoneally bi-weekly (Tuesday-Friday) at 150 mg/kg for twelve weeks as described in Table 6. The dose volume was 30 mL/kg for mice treated intraperitoneally.

TABLE 6
Treatment Groups

| | | | | Dose | Dose | |
|---|---|---|---|---|---|---|
| | | | Dose | Volume | Concentration | Treatment |
| Group | Compound | Route | (mg/kg) | (mL/kg) | (mg/mL) | Frequency |
| 1 | 0.9% NaCl usp | IP | 0 | 30 | 0 | Two consecutive days, |
| | | | | | | stop one day and two |
| | | | | | | other consecutive days |
| | | | | | | for nine weeks |
| 2 | Pentamidine | IP | 45 | 30 | 1.5 | Bi-weekly Monday |
| | | | | | | and Friday for nine |
| | | | | | | weeks |
| 3 | Gemcitabine | IP | 150 | 30 | 5 | Bi-weekly Tuesday |
| | | | | | | and Friday for eleven |
| | | | | | | weeks |
| 4 | Pentamidine | IP | 45 | 30 | 1.5 | Bi-weekly Monday |
| | | | | | | and Thursday for |
| | | | | | | twelve weeks |
| | Gemcitabine | IP | 150 | 30 | 5 | Bi-weekly Tuesday |
| | | | | | | and Friday for twelve |
| | | | | | | weeks |

IP = Intra peritoneally
mg/kg = milligram.kilogram
mL/kg = milliliter per kilogram
mg/mL = milligram per milliliter

At termination, when the tumor volume of one of the mice in the group reached 1500 mm³, the whole group was sacrificed, and animals were anaesthetized using isoflurane and sacrificed by cervical dislocation.

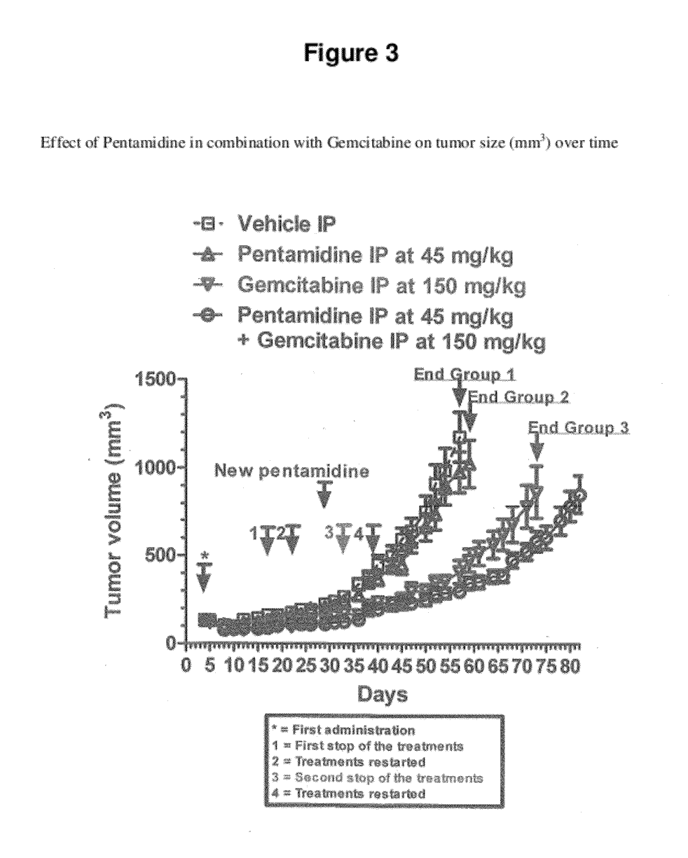

All treatments were well-tolerated. The results are shown in FIG. 3.

To a high degree of statistical confidence, use of pentamidine alone and of pentamidine and gemcitabine in combination were found to have a beneficial effect in the treatment of pancreatic cancer.

1.-21. (canceled)

22. A method of inhibiting the proliferation of cancer cells comprising administering to a patient in need thereof (1) pentamidine and (2) oxaliplatin, gemcitabine, or irinotecan.

23. A composition comprising (1) pentamidine and (2) oxaliplatin, gemcitabine, or irinotecan.

24. A method of claim 22 wherein the amounts of pentamidine and oxaliplatin, gemcitabine, or irinotecan are synergistic.

25. A method of claim 22 wherein the cancer cells are squamous cell carcinoma cells, large cell carcinoma of lymph node cells, breast cancer cells, colon cancer cells, lung carcinoma cells, melanoma cells, pancreatic cancer cells, leukemia cells, non-small cell lung cancer cells, CNS cancer cells, ovarian cancer cells, renal cancer cells or prostate cancer cells.

26. A method of claim 22 wherein the cancer cells are pancreatic cancer cells.

27. A method of claim 22 wherein pentamidine and oxaliplatin are administered.

28. A method of claim 22 wherein pentamidine and gemcitabine are administered.

29. A method of claim 22 wherein pentamidine and irinotecan are administered.

30. A method of claim 28 wherein the cancer cells are colon cancer cells.

31. A method of claim 28 wherein the cancer is localized or metastatic pancreatic cancer.

32. A method of claim 28 wherein the cancer is localized or metastatic breast cancer.

33. A method of claim 27 where the cancer is localized or metastatic colon cancer.

34. A method of claim 29 where the cancer is localized or metastatic colon cancer.

35. A method of treating cancer in a patient comprising administering to said patient (1) pentamidine and (2) oxaliplatin, gemcitabine, or irinotecan, optionally in each case with the further administration of folinic acid, fluorouracil, bevacizumab, cetuximab, panitumumab or a combination thereof.

36. A method of treating pancreatic cancer in a patient comprising administering pentamidine to the patient.

37. A method of claim 36 wherein the cancer is localized or metastatic pancreatic cancer.

38. A method of treating localized or metastatic, pancreatic or ovarian cancer in a patient comprising administering pentamidine to the patient in combination with standard chemotherapy for such cancer.

39. A method of treating ovarian cancer in a patient comprising administering pentamidine to the patient.

40. A method of claim 39 wherein the cancer is localized or metastatic ovarian cancer.

41. A method of treating melanoma, leukemia, non small cell lung cancer, CNS cancer, renal cancer, or prostate cancer in a patient comprising administering pentamidine to the patient.

42. A method of claim 41 wherein the cancer is prostate cancer.

43. A method of inhibiting the proliferation of cancer cells comprising administering to a patient in need thereof (1) pentamidine and (2) taxol or 5-fluorouracil, wherein the cancer cells are melanoma cells, pancreatic cancer cells, leukemia cells, non-small cell lung cancer cells, CNS cancer cells, ovarian cancer cells, renal cancer cells or prostate cancer cells.

44. A method of treating cancer in a cancer patient undergoing second-line chemotherapy comprising administering to said patient:
(1) pentamidine and (2) folinic acid, oxaliplatin, and 5-fluorouracil,
said cancer patient having previously received a first-line chemotherapy comprising folinic acid, 5-fluorouracil and irinotecan;
or administering to said patient:
(1) pentamidine and
(2) folinic acid, 5-fluorouracil and irinotecan,
said cancer patient having previously received a first-line chemotherapy comprising folinic acid, oxaliplatin, and 5-fluorouracil.

45. A method of claim 44 wherein the cancer is localized or metastatic pancreatic cancer, breast cancer, ovarian cancer, colon cancer, melanoma, leukemia, non small cell lung cancer, CNS cancer, renal cancer or prostate cancer.

46. A method of claim 44 wherein the cancer is metastatic colon cancer.

47. A method of claim 44 wherein pentamidine is administered to the cancer patient prior to administering a cycle of folinic acid, oxaliplatin, and 5-fluorouracil or a cycle of folinic acid, 5-fluorouracil and irinotocan.

48. A method of claim 47 wherein pentamidine is administered to the cancer patient on day-2 and/or on day-1 prior to administering a cycle of folinic acid, oxaliplatin, and 5-fluorouracil or a cycle of folinic acid, 5-fluorouracil and irinotocan.

49. A method according to claim 44 wherein pentamidine is administered to the patient at a dose of about 4 mg/kg/day to about 6 mg/kg/day.

50. A method according to claim 49 wherein pentamidine is administered intravenously to the patient at a dose of about 4 mg/kg/day to about 6 mg/kg/day.

51. A method of treating localized or metastatic pancreatic cancer, breast cancer, ovarian cancer, colon cancer, melanoma, leukemia, non small cell lung cancer, CNS cancer, renal cancer or prostate cancer in a cancer patient undergoing second-line or third-line chemotherapy comprising administering to said patient a therapeutically effective amount of pentamidine prior to administering a therapeutically effective amount of standard chemotherapy.

52. A method of claim 51 wherein pentamidine is administered on day-2 and/or on day-1 prior to administering a therapeutically effective amount of standard chemotherapy.

53. A method of claim 51 wherein the cancer is metastatic colon cancer.

54. A method according to claim 51 wherein pentamidine is administered to the patient at a dose of about 4 mg/kg/day to about 6 mg/kg/day.

55. A method according to claim 54 wherein pentamidine is administered intravenously to the patient at a dose of about 4 mg/kg/day to about 6 mg/kg/day.

56. The method according to claim 44 wherein the overall survival of the cancer patient is enhanced when compared with the overall survival associated with standard second-line or third-line chemotherapy.

57. The method according to claim 22 wherein pentamidine is pentamidine isethionate.
